(19) United States
(12) Patent Application Publication
(10) Pub. No.: US 2007/0078386 A1
(43) Pub. Date: Apr. 5, 2007

(54) Movable anchoring catheter

(75) Inventor: Bartolome J. Salazar, Alpharetta, GA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: CYTYC CORPORATION, Marlborough, MA (US)

(21) Appl. No.: 11/215,707

(22) Filed: Aug. 30, 2005

(51) Int. Cl. A61M 29/00

(52) U.S. Cl. 604/96.01

(57) ABSTRACT

Disclosed herein are devices and methods for insertion into a passageway. In one aspect, a device includes a tube having a movable actuator mechanism located at the proximal end thereof and a balloon is disposed on a distal end thereof. The balloon can be coupled to the movable actuator mechanism such that movement of the actuator mechanism results in movement of a portion of the balloon between a first insertion position where the balloon is adapted for insertion into and through the passageway and a second inflation position where the balloon is adapted so as to allow for its inflation.

### FIELD OF THE INVENTION

The present invention relates to catheters configured for insertion into a passageway.

### BACKGROUND OF THE INVENTION

A number of situations arise in which it may be desirable to treat (or to treat other tissue from within) a patient's urethra/ureter with therapeutic rays. One example is the treatment of the prostate. The prostate is a solid organ which surrounds the urethra of the male human between the base of the bladder and the urogenital diaphragm. Benign prostatic hypertrophy (BPH) is a common condition among male humans aged 45 or older. Prostate cancer is a leading cause of death among males, and can frequently be diagnosed with the aid of a simple blood antigen-detecting test. Radiation therapy and prostatectomy are the primary treatments available for prostate cancer and prostatectomy is currently the primary treatment for BPH. Prostatectomy has numerous drawbacks, which have been widely described in the art. External beam irradiation of the prostate for the treatment of localized prostate cancer is associated with small bowel injury, radiation proctitis, and urethral stricture (Gibbons et al., 1979, J. Urol. 121:310-312). At least two groups have employed transurethral radiation therapy as a supplement to external beam irradiation of localized prostate cancer tissue (Harada et al., 1993, Rad. Oncol. 11:139-145; Skarlatos et al., 1994, Urol. Int. 53:209-213). In addition, another group has employed transurethral radiation therapy as a sole treatment for recalcitrant BPH-related urine retention (Koukourakis et al., 1994, Med. Dosimetry 19:67-72). Each of these groups employed ultrasonography, computerized tomography, or fluoroscopy imaging methods to identify the tissue to be treated or to confirm the position of the radiation source relative to the tissue to be treated. Identification of the location of tissue in need of treatment and placement of a radiation source using one of these imaging methods is dependent upon the deformability of the tissues being imaged, the body posture of the subject during the identification or placement, the position of the imaging device, and other factors which may not be easily replicated.

Failure to precisely control the amount and location of transurethrally-delivered radiation can result in damage to the urethra itself or to other organs located in close proximity thereto, including the bladder, rectum and the prostate. It is thus preferable to identify the position of a tissue in need of treatment so that the therapeutic rays can be targeted accurately.

Another example of the delivery of therapeutic rays through the urethra/ureter is the treatment of urethral and ureteric stricture. Urethral stricture is a common complication of urological procedures, particularly following urethral intervention by an urologist (Baskin et al., 1993, J. Urology 150:642-647; Stormont et al., 1993, J. Urology 150:1725-1728). Formation of a urethral stricture is thought to involve disruption of the urothelium, followed by hypertrophy of urothelial or other tissues, resulting in stricture or stenotic lesion. A urethral stricture may also be formed by hypertrophy of a tissue located in close proximity to the urothelium, such as prostate tissue or corpus spongiosum penis tissue in male humans or muscle tissue or spongiose erectile tissue in female humans. Non-limiting examples of urological interventions known to be associated with urethral stricture include transurethral resection of the prostate, radical prostatectomy, external beam irradiation of prostate tissue, and other urological interventions which disturb the urethra. Non-limiting examples of diseases or disorders known to be associated with urethral stricture include BPH, prostate cancer, intemal/external trauma, certain infections and urethral cancer. Further details of tissues which comprise the urethra or which are located in close proximity thereto in the human are found in, for example, Williams et al., eds. (1980, Gray's Anatomy, 36th ed., W.B. Saunders Co., Philadelphia, pp. 1408-1409).

Known treatments for urethral strictures include dilatation of the urethra, endoscopic and open surgical modification of the urethra, laser-assisted modification of the urethra, urethroplasty (e.g., grafts and substitution), and urethral stent implantation (Bosnjakovic et al., 1994, Cardiovasc. Intervent. Radiol. 17:280-284; Badlani et al., 1995, Urology 45:846-856; Mundy, 1989, Brit. J. Urology 64:626-628; Quartey, 1993, Ann. Urol. 27:228-232).

Ureteric stricture is another known complication of urological procedures and of disease and disorder states. Ureteric strictures may involve hyperplasia or hypertrophy of any of the tissue layers of a ureter, namely the fibrous layer, the muscular layer, or the mucous layer, or may involve hyperplasia or hypertrophy of a tissue or organ located in close proximity to a ureter. Further details of tissues which comprise a ureter or which are located in close proximity thereto in the human are found in, for example, Williams et al., eds. (1980, Gray's Anatomy, 36th ed., W.B. Saunders Co., Philadelphia, pp. 1402-1404). Surgical treatments are known for treatment of ureteric stricture.

Bladder neck contracture (BNC) is another condition that can be treated through the urethra and/or bladder. BNC can arise as a complication of urological procedures in which scar tissue forms near the bladder neck and blocks or inhibits the passage of fluid from the bladder. Treatments can include removal of the scar tissue and/or resection of the tissue around the scar tissue.

The prior art teaches several ways for placing devices at a desired location in, for example, the urethra and/or bladder. For example, U.S. Pat. No. 6,607,477 to Longton et al. provides a kit comprising a matched pair of graduated catheters, wherein a graduated locator catheter can provide a known relationship to a position within a bodily lumen. U.S. Pat. No. 5,263,931 to Miller provides a balloon catheter with a core member having a helical coil disposed therearound such that when the balloon is disposed within the patient's prostatic urethra, the coil extends into the patient's bladder. Moreover, U.S. Pat. No. 6,847,848 to Sterzer et al. provides a catheter with an inflatable balloon having a preformed non-uniform, odd-shaped pattern, and U.S. Pat. No. 4,351,342 to Wiita et al. provides a catheter tube having an inflatable balloon mounted on the distal end thereof and ports that open into the balloon such that upon inflation thereof, the inflated balloon completely surrounds the tube port and envelopes the distal end of the tube. While such prior art devices and methods can be effective, they can also be difficult to insert into a passageway, such as the urethra and/or bladder, and time-consuming to use.

A further approach to treating tissue of the urethra is disclosed in U.S. patent application Ser. No. 11/152,824, filed on Jun. 14, 2005 and entitled “Urethral Radiation Treatment and Method,” which is hereby incorporated by reference herein. This application discloses the use of a number of tools to precisely map and treat tissue of the urethra with therapeutic rays. Among the approaches disclosed, is the deployment through a cystoscope of an anchoring element that can seat in a reproducible way in an anatomical feature, such as the bladder neck. The instruments and methods disclosed therein provide for very accurate treatment, however, the cystoscope lumen through which the anchoring element is deployed can be quite narrow, limiting the array of potential anchoring tools that might be deployed in this way.

Accordingly, there remains a need for an improved devices and methods for insertion into a passageway.

### SUMMARY OF THE INVENTION

The present invention provides various methods and devices for insertion of a device into a passageway. In one aspect a balloon catheter is provided that includes a tube having a proximal end with a movable actuator mechanism formed within a handle. A balloon can be disposed on a distal end of the tube and coupled to the movable actuator mechanism by a wire attached at the distal end thereof such that movement of the movable actuator mechanism results in movement of a portion of the balloon. In particular, the movable actuator mechanism is movable between a first insertion position in which the balloon is arranged into a reduced diameter configuration for insertion into and through the passageway, and a second inflation position in which the balloon is arranged so as to allow for its inflation.

While the movable actuator mechanism can have a variety of configurations, in one exemplary embodiment it can be a bias element that can bias the movable actuator to the first, insertion position. Alternatively, the movable actuator mechanism can be a spring-loaded plunger, one or more thumb-operated trigger assemblies, or a screw assembly.

The balloon can be coupled to the movable actuator mechanism in a variety of ways, however in an exemplary embodiment, the proximal end of the balloon can be fixed to the distal end of the tube and the distal end of the balloon can be fixed to a movable tip that is coupled to the movable actuator mechanism. As a result, movement of the movable actuator to the first, insertion position causes the distal tip to move distally to thereby pull the balloon into a reduced diameter configuration.

The device can also include a variety of other features to facilitate use. For example, in one embodiment, the distal end of the tube further comprises a plurality of graduated markers that are adapted to allow a user to measure a depth of the device within a passageway. Alternatively, in another embodiment, the handle can include an input port that is adapted to receive fluid, and the tube have a lumen formed therein that extends into the balloon, such that the lumen can transfer fluid from the input port to the balloon, and the balloon inflate.

In another aspect, a balloon catheter is provided that has an elongate shaft having a handle coupled to a proximal end and a retractable balloon formed on the distal end thereof. The catheter can also include an extension means that is associated with the handle and operatively coupled to the balloon. Activation of the extension means can cause the balloon to move between an extended position to a contracted position. The shaft can further include an inflation means that is adapted to fill the balloon with a fluid such that the diameter of the balloon can increase.

The extension means can have a variety of configurations to effect movement of the balloon between the extended position and the contracted position. In one embodiment, the extension means can include a spring-loaded plunger that is coupled to the balloon by a wire. Alternatively, the extension means can include at least one thumb-operated trigger assembly or a screw system, both of which can be coupled to the balloon by a wire.

Methods for inserting a catheter into a passageway are also provided. In one embodiment, a method includes inserting a tube having a balloon attached to a distal end thereof into a passageway with the balloon in an extended position, and operating an actuator mechanism. The method can also include filling the balloon with a fluid received from an input port in the handle.

The actuator mechanism can be located at a proximal end of the tube and coupled to the balloon such that the balloon moves from the extended position to a contracted position. The actuator mechanism can also have a variety of configurations. In one embodiment, the actuator mechanism can be a plunger having a wire extending therefrom and coupled to the balloon. Thus, the step of operating the actuator mechanism can include releasing the plunger such that the balloon is pulled towards the proximal end of the tube. In another embodiment, the actuator mechanism can include at least one thumb-operated trigger assembly, and the step of operating the actuator mechanism can include releasing the at least one trigger assembly such that the balloon is pulled towards the proximal end of the tube. Alternatively, the actuator mechanism can be a screw system having a wire extending therefrom, and the step of operating the actuator mechanism can include manipulating the screw system such that the balloon is pulled towards the proximal end of the tube.

### BRIEF DESCRIPTION OF THE DRAWINGS

The invention will be more fully understood from the following detailed description taken in conjunction with the accompanying drawings, in which:

FIG. 1A is a perspective view of one exemplary embodiment of a retractable balloon catheter in an insertion position;

FIG. 1B is a perspective view of the retractable balloon catheter of FIG. 1A with the balloon inflated;

FIG. 2 is a cross-sectional view of the distal end of the retractable balloon catheter shown in FIG. 1A;

FIG. 3 is a cross-sectional view of the distal end of the retractable balloon catheter shown in FIG. 1B;

FIG. 4 is a cross-sectional view of the proximal end of the retractable balloon catheter shown in FIG. 1A;

FIG. 5 is a cross-sectional view of the proximal end of the retractable balloon catheter shown in FIG. 1B; and

FIG. 6 is cross-sectional view of the proximal end of the retractable balloon catheter showing the flow of fluid through the fluid input port.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are devices and methods for inserting a surgical tool into a passageway. While the devices and methods disclosed herein can be used in a variety of bodily pathways, such as the stoma, carotid arteries, peripheral vessels, urethra, esophagus, and bile duct, in an exemplary embodiment the device is configured for insertion through the urethra for anchoring of the device within the bladder. Still further, the devices and method disclosed herein may be used to place an anchoring element through narrow lumen in another surgical tool, such as through a lumen in a cystoscope that is deployed in a patient's urethra as described in the above-incorporated U.S. patent application Ser. No. 11/152,824. However, a person skilled in the art will appreciate that the methods and devices disclosed herein can have a variety of configurations, and they can be adapted for use in a variety of medical procedures require insertion of a device through a passageway.

In general the invention includes a device that has a distal anchoring element that can be manipulated between a first, insertion position, in which the anchoring element is “stowed” or easier to insert through a narrow bodily passage, and a second, anchoring position in which the anchoring element can be deployed to anchor the device in a desired position within a patient's body. The device preferably includes a catheter extending proximally from the anchoring element to a proximal end portion that would extend outside of the patient's body. An actuator on the proximal portion, and thus outside the patient's body, can be acted upon to manipulate the anchor.

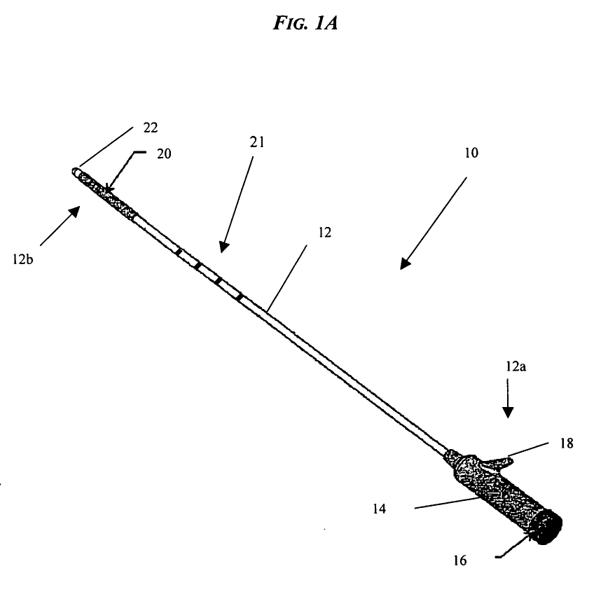

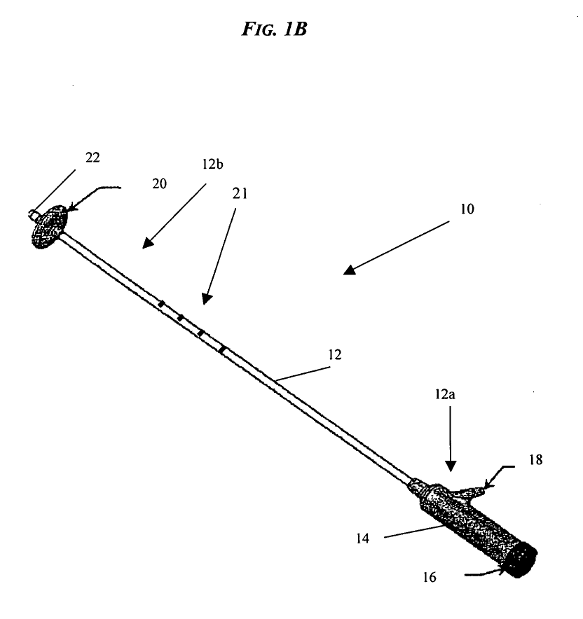

FIG. 1A-1B illustrate one exemplary embodiment of a device that can be inserted into a passageway. In this exemplary embodiment, the anchoring element is a balloon 20, shown in the first, insertion position in FIG. 1A and in the second, anchoring position in FIG. 1B. Extending proximally from the balloon is a catheter tube 12 having proximal and distal ends 12a, 12b. The proximal end 12a of the tube 12 can have a handle 14 with a movable actuator mechanism 16 located therein. Balloon 20, disposed on the distal end 12b of the tube 12, can be coupled to the movable actuator mechanism 16 such that movement of the actuator mechanism 16 results in movement of a portion of the balloon 20 so that the balloon is moved between a first insertion position in which the balloon 20 is adapted for insertion into and through the passageway (shown in FIG. 1A), and a second inflation position in which the balloon 20 is adapted so as to allow for its inflation (shown in FIG. 1B).

Tube 12 as illustrated has a substantially cylindrical shape, however, it can have a variety of cross-sectional configurations as needed, such as circular, oval, ellipsoid, kidney-bean shaped, a composite of substantially circular, oval, or ellipsoid forms, or irregularly shaped.

The tube 12 can also have a variety of sizes depending upon its desired use, however generally the tube 12 can have a length in the range of about 20 to 60 cm. Generally useful diameters of such a catheter would range from about 0.25 mm to 3 mm, though larger diameters may have utility (e.g., up to 2 cm in diameter). In an exemplary embodiment, where the tube 12 is adapted for placement within the urinary tract the tube can be adapted such that at least a portion of the proximal end 12a of the tube 12 can extend outwardly from the urinary tract after the tube 12 has been inserted into the patient, and have a length that can be greater than about 20 centimeters and a diameter of about 1 to 2 mm.

The tube can be made of a material that is sufficiently rigid such that it may be urged into the urethra of a patient without kinking, but sufficiently pliable that it will follow the curvature of the patient's urethra. In exemplary embodiment, the tube can be made of a biocompatible plastic or elastomer. Suitable biocompatible plastics include materials such as polyethylene, a homopolymer or copolymer of vinyl acetate such as ethylene vinyl acetate copolymer, polyvinylchloride, a homopolymer or copolymer of acrylate such as polymethylmethacrylate, polyethylmethacrylate, polymethacrylate, ethylene glycol dimethacrylate, ethylene dimethacrylate and hydroxymethyl methacrylate, polyurethane, polyvinylpyrrolidone, 2-pyrrolidone, polyacrylonitrile butadiene, polycarbonate, polyamide, a fluoropolymer such as a homopolymer or copolymer of polytetrafluoroethylene and polyvinyl fluoride, polystyrene, a homopolymer or copolymer of styrene acrylonitrile, cellulose acetate, a homopolymer or copolymer of acrylonitrile butadiene styrene, polymethylpentene, polysulfone, polyester, polyimide, polyisobutylene, polymethylstyrene, and other similar compounds known to those skilled in the art. Suitable biocompatible elastomers include, for example, silicone rubber, polyvinyl chloride elastomer, polyolefin homopolymeric or copolymeric elastomer, urethane-based elastomer, one of the class of polyurethanes, latex, or synthetic rubber.

The tube can also have a variety of other features to facilitate insertion into a patient. For example, the tube can have features to help provide support thereto and prevent kinking thereof. While these features can vary, in one embodiment, the tube can have ribs formed therein or therearound made out of any of the aforementioned materials. Alternatively, the tube can have a wire or a spring extending along the length thereof. Moreover, the movable tip 22 formed at the distal end of the tube 12 (which will be discussed in more detail below) can also be configured for insertion into a passageway. For example the movable tip 22 can be rounded and have a diameter that is similar to that of the tube 12, and/or have a distal taper to facilitate insertion into the pathway. One skilled in the art will appreciate the variety of other features that the tube can have to facilitate insertion or positioning into a passageway.

The tube can also include graduated markers 21 formed on least at a distal portion thereof to assist in positioning the tube within the passageway. For example, fiducial markers, such as those disclosed in patent application Ser. No. 10/704,161, entitled “Tissue Positioning Systems and Methods for Use with Radiation Therapy,” and incorporated herein by reference in its entirety, can be used with the tube. Such fiducial markers can be used according to the methods disclosed in the previously incorporated U.S. patent application Ser. No. 11/152,824 to map a body lumen for the purpose of providing an accurate delivery of therapeutic rays to a target tissue in the lumen. In addition, radiation sensors can be used with or disposed on tube to assist with delivery of therapeutic radiation. Utility application Ser. No. 10/704,340, entitled, “Implantable Radiotherapy/Brachytherapy Radiation Detecting Apparatus and Method,” and incorporated herein by reference in its entirety, discloses exemplary sensors.

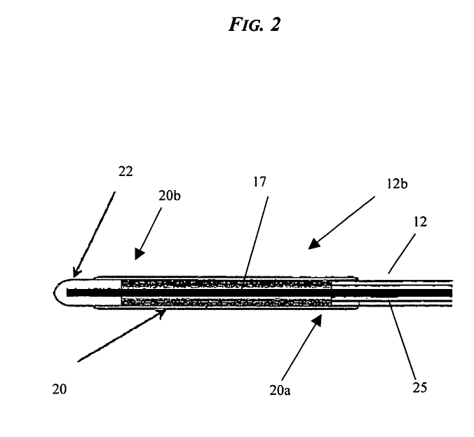

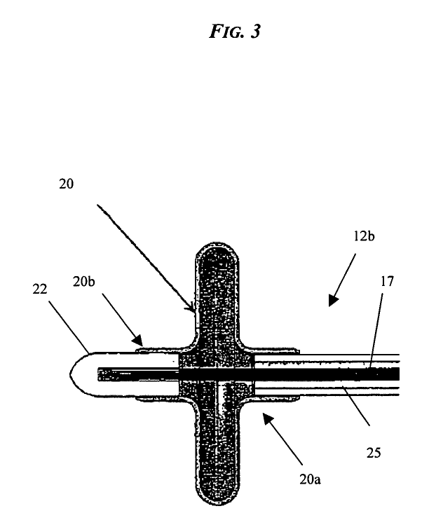

Referring now to FIGS. 2 and 3, the balloon 20 can be fixed on a portion of the distal end 12b of the tube 12 and adapted to extend (FIG. 2) and contract (FIG. 3) in response to actuation by the actuation mechanism 16, as will be discussed in more detail below. Once contracted, the balloon can also be adapted to inflate to a desired size. In the illustrated embodiment, a proximal end 20a of the balloon 20 is fixed to a distal end of the tube 12, while a distal end 20b of the balloon 20 is fixed to distal tip 22. The balloon 20 can be extended into its insertion portion by pushing distal tip 22 distally, for example by wire 17 extending through lumen 25 of tube 12 and through the balloon 20 to the distal tip. The balloon 20 can be retracted into its contracted or inflation position by drawing back wire 17 to move the distal tip 22 proximally as shown in FIG. 3.

While the balloon 20 can have a variety of configurations, in an exemplary embodiment it can be complementary in shape and diameter to the tube 12 when stretched in an extended position. The balloon 20 can also have a variety of sizes, and in an exemplary embodiment, the balloon 20 can have an inflated diameter that is preferably in the range of about 1.0 to 3.0 cm for use in the urethra. Uninflated, its diameter would be substantially equal to that of the catheter shaft. For any given application, the inflated or expanded diameter would be at least 50% larger than the average diameter of the body cavity opening into which it is inserted.

One skilled in the art will appreciate that the balloon can be made of a variety of materials, however in an exemplary embodiment is made from a material that allows it to extend and contract in response to the movable actuator mechanism, such as a biocompatible elastomer. Suitable biocompatible elastomers include silicone rubber, polyvinyl chloride elastomer, polyolefin homopolymeric or copolymeric elastomer, urethane-based elastomer, latex, or synthetic rubber, and any other known in the art.

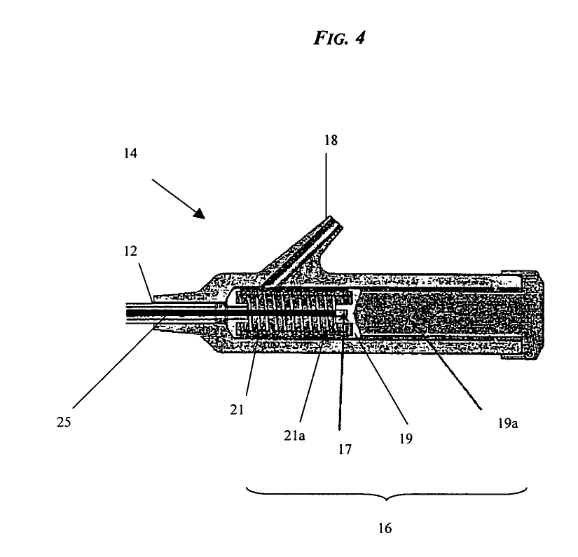

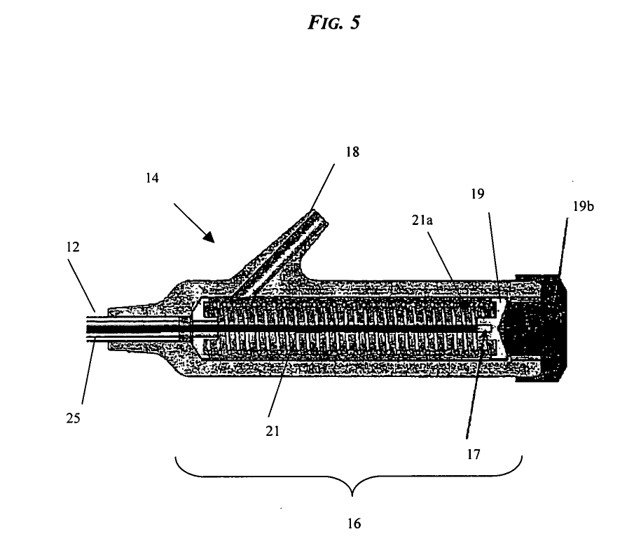

The balloon 20 can be actuated using a variety of movable actuator mechanisms 16. Preferably, the actuator mechanism is located on a proximal portion of the device that extends outside the patient during its use so that a surgeon can manipulate the anchoring element from outside the patient while the anchoring element is being deployed within the patient. In one exemplary embodiment illustrated in FIGS. 4 and 5, the movable actuator mechanism 16 can be a spring-loaded plunger located in the handle 14 on the proximal end 12a of the tube 12. In the illustrated embodiment, the movable actuator mechanism 16, or spring-loaded plunger, can include a plunger 19 and a spring 21 positioned within the handle 14. Upon the application of force to the plunger 19, the plunger 19 can contact the spring 21 and cause it to contract, thereby pushing wire 17 to extend distal tip 22 (FIGS. 1-3) and thereby effect actuation of the balloon 20 into its first, insertion position (illustrated in FIG. 2).

As illustrated in FIG. 4, handle 14 includes an extension or insertion plunger 19a. The extension plunger 19a is screwed onto a proximal end of the handle 14 and has a length sufficient to push plunger 19 to a position that will move the balloon 20 into its first, insertion position. After the device has been inserted to a passageway to a point where it is desired to deploy the balloon as an anchoring element, the extension plunger 19a is unscrewed and removed. At this point, spring 21 will act to push plunger 19 proximally to thereby retract the distal end of the balloon, moving the balloon to its second, inflation position. In order to seal the device so that the balloon can be inflated, a retraction or inflation plunger 19b, shown in FIG. 5, can be screwed onto handle 14 in place of extension plunger 19a. The retraction plunger 19b has a length that allows the spring 21 to hold the plunger 19 up against the retraction plunger 19b in a position that corresponds to the inflation position of the balloon.

Other exemplary movable actuator mechanisms may include one or more trigger assemblies that can be thumb-operated, a screw assembly formed within the handle 14, or other forms of sliding actuators as may be known in the art that can move the movable actuator between a first, insertion position and a second inflation position.

The balloon can be coupled to the actuation mechanism 16 using a variety of techniques. In the illustrated embodiments, the proximal end 20a of the balloon 20 can be fixed to the distal end 12b of the tube 12, and the distal end 20b of the balloon 20 can be fixed to a movable tip 22 that is coupled to the movable actuator mechanism 16. A wire 17 can be coupled to the movable tip 22, extend along the length of the tube 12, and be coupled to the movable actuator mechanism 16 to effect actuation of the balloon 20. In particular, the wire 17 is coupled to the proximal end 21a of the spring 21. This allows the wire 17 to receive the extension force from the plunger 19, and push the balloon 20 to an extended position. One skilled in the art will appreciate that the movable actuator mechanism can be coupled to the balloon in a variety of other ways, for example, the wire can be connected to the distal end of the balloon itself, or, alternatively, proximal end of the balloon may be moved to place the balloon in insertion or inflation positions.

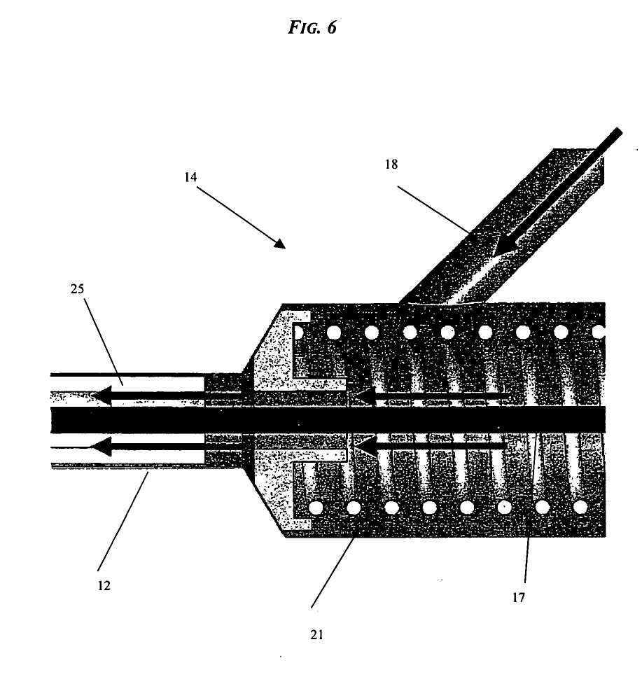

As illustrated in FIGS. 4 and 5, and also more particularly in FIG. 6, the handle 14 can also include at least one fluid input port 18 integrally formed therein and adapted to receive fluid, or alternatively, gas, to cause the balloon 20 to inflate. While the input port 18 can have a variety of configurations, it is preferably in communication with lumen 25 formed along the length of the tube 12 and opening at the proximal end 20a of the balloon such that upon the receipt of fluid therein, the balloon 20 can inflate. The input port 18 can also be adapted to connect to an external fluid source in a variety of ways as are known in the art. Further, while the device as illustrated includes a single lumen for passing wire 17 as well as for inflating balloon 20, and this feature is advantageous in making the illustrated embodiment capable of passing through narrow passageways, a person of ordinary skill in the art will recognize that separate or additional lumens could be added depending on the desired use of the device.

As noted above, the device can be used to effect insertion into a passageway of a patient. In one exemplary embodiment, the catheter can be advanced through a pathway in a patient's body, such as the urethral canal, until the balloon is positioned at the bladder neck with the balloon in an insertion or extended position. The device can also be inserted into the passageway of another instrument, such as through a lumen in a cystoscope as described in the previously incorporated U.S. patent application Ser. No. 11/152,824. In order to effect the insertion position, as shown in FIGS. 1A, 2 and 4, a user can apply pressure to the plunger 19 such that the plunger 19 contacts the spring 21 and causes the spring 21 to contract. The contraction of the spring 21 causes the wire 17 and movable tip 22 attached thereto to be pushed in a distal direction, such that the balloon 20 is stretched or extended along a length of the tube 12, as shown in FIG. 2. This pressure can be applied using the extension plunger 19a. When the balloon 20 is in the insertion position, it has a diameter that is substantially similar to that of the tube 12, thereby making the balloon 20 easier to insert through the passageway.

Once the device is at the desired position within or through the passageway, the balloon 20 can be moved into an inflation or retracted/contracted position as shown in FIGS. 1B, 3, and 5. This can be effected by releasing the pressure on the plunger 19. As a result of the pressure release, the spring 21 can expand in the proximal direction, pulling the wire 17 and the movable tip 22 along with it such that at least a portion of the balloon 20 is contracted. This increased diameter configuration allows the balloon 20 to receive fluid or gas to effect inflation thereof. The balloon 20 can be inflated to a desired diameter with a fluid received from an input port 18 in the handle 14, as shown in FIGS. 4-6. While the balloon 20 can be filled to a variety of amounts, in an exemplary embodiment, the balloon 20 can inflate such that the dimensional ratio between the balloon 20 in an uninflated state and the balloon in an inflated state is about 0.05 to 0.20.

One skilled in the art will further appreciate features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A balloon catheter system for insertion into a passageway, comprising:
a tube having proximal and distal ends, the proximal end having a handle coupled thereto;
a movable actuator mechanism located on the handle; and
a balloon disposed on a distal portion of the tube and coupled to the movable actuator mechanism so that movement of the movable actuator mechanism results in movement of a portion of the balloon;
wherein the movable actuator mechanism is movable between a first insertion position in which the balloon is arranged into a reduced diameter configuration for insertion into and through the passageway, and a second inflation position in which the balloon is arranged so as to allow for its inflation.

2. The system of claim 1, wherein the movable actuator mechanism includes a bias element.

3. The system of claim 1, wherein the balloon is coupled to the movable actuator mechanism by a wire attached at a distal end thereof.

4. The system of claim 3, wherein a proximal end of the balloon is fixed to the distal end of the tube and the distal end of the balloon is fixed to a movable tip that is coupled to the movable actuator mechanism.

5. The system of claim 4, wherein movement of the movable actuator to the first, insertion position causes the distal tip to move distally to thereby pull the balloon into a reduced diameter configuration.

6. The system of claim 1, wherein the movable actuator mechanism is a spring-loaded plunger.

7. The system of claim 1, wherein the movable actuator mechanism includes one or more trigger assemblies that can be thumb-operated.

8. The system of claim 1, wherein the movable actuator mechanism is a screw assembly.

9. The system of claim 1, wherein the distal end of the tube further comprises a plurality of graduated markers that are adapted to allow a user to measure a depth of said system within a passageway.

10. The system of claim 1, wherein the handle further comprises an input port that is adapted to receive fluid, and the tube has a lumen formed therein and extending into the balloon, such that the lumen can transfer fluid from the input port to the balloon, and the balloon inflate.

11. The system of claim 1, wherein the system is configured for insertion through the urethra for inflation of the balloon within the bladder.

12. The system of claim 1, wherein in the first insertion position, the balloon is arranged into a reduced diameter configuration for insertion into and through the lumen of a medical device.

13. The system of claim 12, further comprising a medical device having a lumen, the balloon in its first insertion position being arranged for insertion into the lumen.

14. The system of claim 13, wherein the medical device is one selected from the group consisting of a cystoscope and an endoscope.

15. A balloon catheter, comprising:
an elongate shaft having proximal and distal ends, the proximal end being coupled to a handle and the distal end having a retractable balloon formed thereon; and
an extension means associated with the handle and operatively coupled to the balloon, wherein activation of the extension means causes the balloon to move between an extended position to a contracted position.

16. The catheter of claim 15, wherein the extension means includes a spring-loaded plunger that is coupled to the balloon by a wire.

17. The catheter of claim 15, wherein the extension means includes at least one thumb- operated trigger assembly that is coupled to the balloon by a wire.

18. The catheter of claim 15, wherein the extension means includes a screw system that is coupled to the balloon by a wire.

19. The catheter of claim 15, wherein the shaft further comprises an inflation means that is adapted to fill the balloon with a fluid such that the diameter of the balloon can increase.

20. A method for inserting a catheter into a passageway, comprising:
inserting a tube having a balloon attached to a distal end thereof into a passageway with the balloon in an extended position; and
operating an actuator mechanism located at a proximal end of the tube and coupled to the balloon such that the balloon moves from the extended position to a contracted position.

21. The method of claim 20, wherein the actuator mechanism is a plunger having a wire extending therefrom and coupled to the balloon, and wherein operating the actuator mechanism includes releasing the plunger such that the balloon is pulled towards the proximal end of the tube.

22. The method of claim 20, wherein the actuator mechanism includes at least one thumb-operated trigger assembly, and operating the actuator mechanism includes releasing the at least one trigger assembly such that the balloon is pulled towards the proximal end of the tube.

23. The method of claim 20, wherein the actuator mechanism is a screw system having a wire extending therefrom, and operating the actuator mechanism includes manipulating the screw system such that the balloon is pulled towards the proximal end of the tube.

24. The method of claim 20, further comprising filling the balloon with a fluid received from an input port in the handle, such that the balloon inflates.

25. The method of claim 24, wherein the balloon is filled with fluid such that the dimensional ratio between the balloon in an uninflated state and the balloon in an inflated state is about 0.5 to 0.20.

26. The method of claim 20, wherein the passageway is a lumen in a medical device.

27. The method of claim 26, wherein the medical device is one selected from the group consisting of a cystoscope and an endoscope.
